(19) United States
(12) Patent Application Publication
(10) Pub. No.: US 2011/0144591 A1
(43) Pub. Date: Jun. 16, 2011

(54) Transdermal Delivery Device

(76) Inventor: Russell F. Ross, Atlanta, GA (US)

(21) Appl. No.: 12/636,281

(22) Filed: Dec. 11, 2009

(51) Int. Cl. A61M 5/32

(52) U.S. Cl. 604/173

(57) ABSTRACT

The present invention relates generally to a transdermal delivery device which is suitable for the transdermal delivery or removal of substances, and in particular relates to a transdermal delivery device having a support and a plurality of microneedles projecting outwardly from the support, at least one microneedle including a channel positioned on the exterior surface which aligns with at least one aperture being formed in the support.

### BACKGROUND OF THE INVENTION

Numerous products are available which deliver therapeutic substances through the skin of a user using a plurality of very small needles assembled into a device. These microneedles are generally slender elongated shafts that have sufficient length to enable the tip of the structure to penetrate the stratum corneum layer of the skin and pass into the epidermal layer of the skin. Exemplary devices are disclosed in U.S. Pat. No. 6,881,203, WO 2007/0260201 and U.S. Pat. No. 3,964,482. Devices including microneedles have been useful in the movement of substances such as drugs through the skin barrier in a relatively painless yet effective manner by providing minimal trauma and pain at the delivery site by precise control of the depth of penetration of the microneedles. Such products are also useful in the removal through the skin of substances for analysis, such as, for example, blood and tissue.

Microneedles may be formed having a hollow shaft, similar to larger conventional medical needles, so that substances may be delivered or withdrawn through the hollow shaft. Microneedles having this configuration are particularly suitable for use with micropumps which are able to precisely control the amount of substance delivered through each device. However, due to their very small size, the hollow shafts may break off in use or become easily blocked as the substance moves through the full length of the hollow shaft.

Other microneedles may have one or more channels on the exterior surface of the shaft. These exterior channels have fewer tendencies to become blocked. However, devices including such microneedles may not provide sufficient control over the quantity of substance to be delivered. This can be particularly important when such devices are utilized to deliver drugs.

As a result, there exists a need for a transdermal delivery device which permits adequate control over the quantity of substance delivered or removed while reducing the opportunities for breakage and/or blockages.

### SUMMARY OF THE INVENTION

In accordance with one embodiment of the present invention, a transdermal delivery device is provided, the device including a support having a first surface and a second surface. A plurality of microneedles are positioned on and project outwardly from the second surface of the support. At least one microneedle includes a base, a tip and an exterior surface. A pathway for fluid to pass through the transdermal delivery device is provided, the pathway including an aperture which extends between the first surface of the support and the second surface of the support. The pathway also includes a channel disposed on the exterior surface of the microneedle, the channel being in alignment with at least a portion of the aperture to form a junction through which substances may pass. The junction is typically formed in the plane of the second surface at the base of the microneedle.

In selected embodiments, the junction may have a cross-sectional area that is greater than or equal to about 100 square microns. In particular embodiments having a plurality of channels and junctions on a single microneedle, the total cross-sectional area of all junctions may be greater than or equal to about 300 square microns.

In some transdermal delivery devices, the microneedle may have a channel that has a cross-sectional area, measured proximate to the base of the microneedle, that is in the range of from about 0.5% to about 40%, and in selected microneedles may range from about 5% to about 30%, and in other microneedles may range from about 10% to about 25%. In selected microneedles containing a plurality of channels, similar ranges may be pertinent for the total cross-sectional area of all channels. Additionally, percentages different from these exemplary ranges may also be suitable for use in the present invention.

In accordance with another embodiment of the present invention, a transdermal delivery device is provided that includes a support having a first surface, a second surface and at least one aperture extending through the first surface and the second surface. A plurality of microneedles project outwardly from the second surface of the support, and at least one microneedle has a base, a tip, and an exterior surface. At least one channel is positioned on the exterior surface of at least one microneedle, the channel extending to the base of the microneedle. A junction is formed in the plane of the second surface at the base of the microneedle by the intersection of the aperture and the channel. In some embodiments, the junction may have a cross-sectional area that is greater than or equal to about 100 square microns. In some embodiments, the cross-sectional area of a channel proximate to the base of the microneedle is greater than or equal to about 100 square microns. In embodiments which include at least two microneedles, each microneedle having at least one junction, the total cross-sectional area of the junctions is greater than or equal to about 600 square microns.

Other features and aspects of the present invention are described in more detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a perspective view of a portion of a transdermal delivery device in accordance with an embodiment of the present invention;

FIG. 2 is a cross-sectional view of a portion of a transdermal delivery device of FIG. 1, taken along lines 2-2;

FIG. 3 is a top view of a portion of a transdermal delivery device that may be formed in accordance with an embodiment of the present invention;

FIG. 4 is a bottom view of a portion of a transdermal delivery device that may be formed in accordance with an embodiment of the present invention;

FIGS. 5 and 6 are partial cross-sectional views of transdermal delivery devices that may be formed in accordance with an embodiment of the present invention;

FIG. 7 is a cross-sectional view of a microneedle in accordance with an embodiment of the present invention; and

FIG. 8 is a top view of a portion of another transdermal delivery device that may be formed in accordance with an embodiment of the present invention.

Repeat use of reference characters in the present specification and drawings is intended to represent same or analogous features or elements of the invention.

### DETAILED DESCRIPTION OF REPRESENTATIVE EMBODIMENTS

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention cover such modifications and variations.

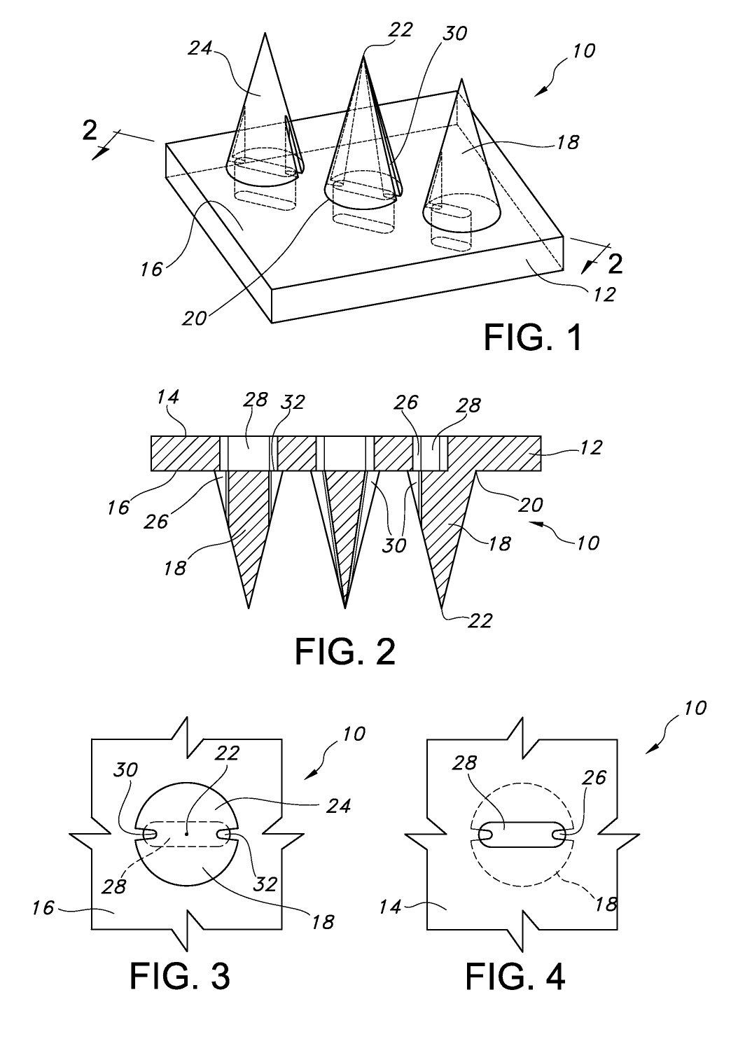

The present invention is generally directed to a transdermal delivery device 10, a portion of which is depicted in FIG. 1. The transdermal delivery device 10 includes at least one microneedle 18 which extends from a support 12. The support 12 may include a first surface 14 and a second surface 16. The support 12 may be constructed from a rigid or flexible sheet of metal, ceramic, plastic or other material. The support 12 can vary in thickness to meet the needs of the transdermal delivery device. In some embodiments, the support 12 is about 1000 microns or less, while in other embodiments the support 12 may be 500 microns or less. The support 12 may also be formed of a substrate which is relatively thin, such that the support 12 is 200 microns or less.

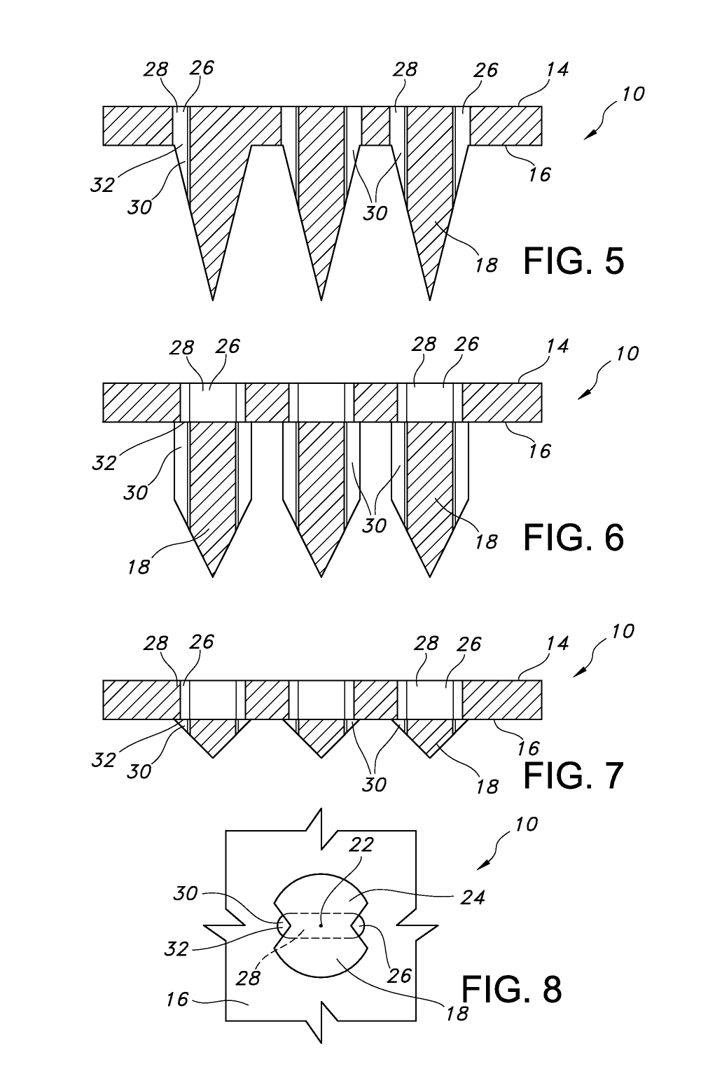

An aperture 28 is formed in the support 12 such that the aperture 28 extends through the first surface 14 and through the second surface 16. In the embodiment depicted in FIGS. 1 and 2, the microneedles 18 extend from the second surface 16, although in other embodiments the microneedles 18 may extend from the first surface 14 or elsewhere. The microneedles 18 of FIGS. 1 and 2 have an overall conical shape, although the microneedles 18 may have any of a variety of overall shapes. For example, the microneedles 18 may have an overall pyramidal shape or a cylindrical portion upon which is positioned a conical portion having a tip, such as is shown in FIGS. 5 and 6.

The microneedle 18 preferably includes a base 20, a tip 22 and an exterior surface 24. As shown in FIG. 1, the base 20 is the portion of the microneedle 18 that is proximate to the second surface 16 of the support 12. The tip 22 of the microneedle 18 is the point of the microneedle 18 which is furthest from the base 20. Although the tip 22 may be variously formed, the tip 22 of the microneedle 18 may have a radius that is less than or equal to about 1 micron.

The microneedles 18 should be sufficiently long to penetrate the stratum corneum and pass into the epidermis. Preferably, the microneedles should not penetrate through the epidermis and into the dermis in applications where it is desirable to minimize pain. In selected embodiments, the microneedles may be 500 microns or less in length (from their tip 22 to their base 20), and in particular embodiments may be 250 microns or less in length. The diameter of the microneedle 18 may vary along the length of the microneedle 18, and may range from 250 microns or less, and in other embodiments may range from about 125 microns or less.

A channel 30 is positioned on the exterior surface 24 of the microneedle 18. A pathway 26 is formed by the channel 30 and the aperture 28, which meet at a junction 32 that is generally located in the plane of the second surface 16. Each microneedle 18 may deliver or extract substances through the skin via the pathway 26, as depicted in FIG. 2. The pathway 26 enables a substance to flow from the first surface 14 through the aperture 28, the junction 32 and exiting into the channel 30. By enabling the substance to flow through the support 12 and directly into the channel 30, more precise control over the delivery location and the amount of substance delivered may be provided.

In selected embodiments and as shown in FIG. 5, an aperture 28 is aligned with a single channel 30 via a junction 32. Alternately and as shown in other figures, a single aperture may feed two or more separate channels 30.

The dimensions of the support, microneedle, apertures, channels and junctions will be interdependent and may vary substantially, depending on the desired use of the transdermal delivery device 10. For example, a conical microneedle 18 having a diameter at its base of about 120 microns and a height of at least 150 microns may include at least two channels 30. Each channel in such a microneedle 18 may have a depth at the base 20 of approximately 40 microns. The depth of the channel 30 may, in selected embodiments, vary along the length of the channel. In certain embodiments, the channel 30 will be deeper proximate to the base of the microneedle than proximate to the tip 22 of the microneedle 18. The channels 30 in this example may have v-shaped or u-shaped cross-sections, as seen in FIGS. 3, 4 and 8. The channels 30 may, in this example, have a cross-sectional area proximate to the base of the microneedle of at least about 250 square microns each. In such an example, each junction 32 may be approximately 150 square microns.

A mechanism may be provided to move a substance through the transdermal delivery device 10. Selected substances such as drugs may require precise control of the quantity of substance delivered via the microneedles 18. A fluid reserve may be provided adjacent to the first surface 14 of the support 12 in selected embodiments. A pump, such as mechanical, thermal, electrical, chemical or other pumping mechanisms may be provided to move a substance through the microneedle 18.

The channel 30 may extend from the junction 32 at the base 20 of the microneedle to the tip 22, as depicted in FIGS. 1 and 2. In other embodiments, the channel 30 may not extend the full length of the microneedle 18 to the tip 22. Each microneedle 18 may include more than one channel 30, as seen in the embodiments of FIGS. 5, 6 and 7. Alternate embodiments may include more channels if desired. In some embodiments, six channels may be utilized. The channel 30 may be variously positioned on the exterior surface 24, forming a substantially linear path from the base 20 towards the tip 22, or forming a winding or circuitous path along the exterior surface 24. In microneedles where two or more channels are present, the channels 30 may be variously spaced around the microneedle 18 in a symmetrical or asymmetrical manner.

FIG. 4 is a view looking at the first surface 14 of the transdermal delivery device 10 which may be proximate to the pumping mechanism, and shows the junction 32 that is formed in the pathway 26 by the overlapping portions of the aperture 28 and the channel 30. FIG. 3 is a view looking down onto the second surface 16 of the microneedle 18, showing the junction 32 as seen from that portion of the transdermal delivery device 10 which may be in contact with the skin of a user. The junction 32 may vary in area between pathways 26 on a given microneedle 18, and may vary between microneedles 18 on a given device 10. The area of the junction 32 may vary widely, and will depend on factors such as, for example, the diameter of the microneedle 18, the viscosity of the substance to be moved through the pathway 26 and the quantity of substance to be delivered. In selected embodiments, the area of the junction 32 at the second surface 16 is greater than or equal to about 100 square microns, although smaller areas may also be acceptable for use in the present invention. In other embodiments, the area of the junction 32 at the second surface 16 may be equal to about 150 square microns or greater.

The cross-section of the channel 30, as shown in FIG. 7, is substantially u-shaped. The channel 30 may also be arcuate or have any other configuration suitable for moving a substance therethrough, such as, for example, v-shaped or c-shaped. The channel 30 may also change shape or cross-section along its length and/or width.

In particular embodiments, it is desirable to determine the cross-sectional area of the channel 30 as a percent of the cross-sectional area of the microneedle 18 proximate to the base 20 at the second surface 16. While this calculation can be performed in various manners, it is preferable that the cross-sectional area of the base 20 first be determined assuming that the channel 30 is not present. The cross-sectional area of the channel 30 may then be determined. To calculate the percent cross-sectional area of the channel 30, the cross-sectional area of the channel 30 at the base 20 is multiplied by 100, then divided by the cross-sectional area of the microneedle 18 at its base 20, assuming that the channel 30 is not present.

FIG. 5 illustrates embodiments of the microneedle 18 in which the aperture 28 and channel 30 have sides which are not only coextensive with each other but may also be planar for at least some distance along the length of the pathway 26. FIGS. 6 and 7 illustrate an embodiment where a single aperture 28 is aligned with more than one channel 30 on a particular microneedle 18. FIG. 8 is a view of the second surface 16 of the device 10 which is shown in FIG. 7, illustrating the alignment of the microneedle 18, the channels 30, the aperture 28 and the junctions 32.

The microneedles 18 may be arranged on the substrate in a variety of patterns, and such patterns may be designed for a particular use. For example, the microneedles may be spaced apart in a uniform manner, such as in a rectangular or square grid or in concentric circles. Spacing between the microneedles 18 may depend on numerous factors, including height and width of the microneedles 18 as well as the amount and type of substance that is intended to be moved through the microneedles. While a variety of arrangements of microneedles is useful in the present invention, a particularly useful arrangement of microneedles 18 is a tip-to-tip spacing between microneedles of at least about 100 microns, and more preferably at least about 300 microns.

Microneedles 18 may be formed of various substances such as, for example, polymers, ceramics and metals. While numerous processes may be used to manufacture microneedles according to the present invention, a suitable production system is MEMS (Micro-Electro-Mechanical Systems) technology and microfabrication processes. MEMS is capable of forming micromechanical and other elements such as semiconductors on a single silicon substrate using microfabrication processes such as etching, micromachining or other processes. The substrate 12 may be manufactured from silicon, the microneedles being subsequently formed by a microetching process. Micromolding techniques may also be used to form the microneedles 18 and support 12 of the present invention.

While the invention has been described in detail with respect to the specific embodiments thereof, it will be appreciated that those skilled in the art, upon attaining an understanding of the foregoing, may readily conceive of alterations to, variations of, and equivalents to these embodiments. In addition, it should be noted that any given range presented herein is intended to include any and all lesser included ranges. For example, a range of from 45-90 would also include 50-90; 45-80; 46-89 and the like. Accordingly, the scope of the present invention should be assessed as that of the appended claims and any equivalents thereto.

What is claimed is:

1. A transdermal delivery device comprising:
a support comprising a first surface and a second surface;
a plurality of microneedles projecting outwardly from the second surface of the support, at least one microneedle comprising a base, a tip and an exterior surface;
a pathway comprising
an aperture extending between the first surface of the support and the second surface of the support;
a channel disposed on the exterior surface of at least one microneedle, the channel at the base being in alignment with at least a portion of the aperture to form a junction through which substances may pass, the junction being formed in the plane of the second surface at the base of the microneedle.

2. The transdermal delivery device as claimed in claim 1, the junction having a cross-sectional area that is greater than or equal to about 100 square microns.

3. The transdermal delivery device as claimed in claim 1, wherein the junctions on at least one microneedle, when added together, have a total cross-sectional area that is greater than or equal to about 300 square microns.

4. The transdermal delivery device as claimed in claim 1, the channel having a cross-sectional area proximate to the base of the microneedle that is less than or equal to about 25% of the total area of the base.

5. The transdermal delivery device as claimed in claim 1, the channel having a cross-sectional area proximate to the base of the microneedle that is less than or equal to about 0.5% of the total area of the base.

6. The transdermal delivery device as claimed in claim 1, wherein at least one microneedle has at least two channels, the total cross-sectional area of the channels proximate to the base of the microneedle being less than or equal to about 40% of the total area of the base.

7. The transdermal delivery device as claimed in claim 1, the tip of the microneedle having a radius of less than or equal to about one micron.

8. The transdermal delivery device as claimed in claim 1, the microneedle having a conical shape proximate to the tip of the microneedle.

9. The transdermal delivery device as claimed in claim 1, the channel forming a non-linear path on the exterior surface of the microneedle.

10. A transdermal delivery device comprising:
a support comprising a first surface, a second surface and at least one aperture extending through the first surface and the second surface;
a plurality of microneedles projecting outwardly from the second surface of the support, at least one microneedle comprising
a base having a cross-sectional area,
a tip, and
an exterior surface, at least one channel being disposed on the exterior surface of at least one microneedle, the at least one channel extending to the base of the microneedle;
a junction being formed in the plane of the second surface at the base of the microneedle by the intersection of the at least one aperture and the at least one channel, the junction having a cross-sectional area that is greater than or equal to about 100 square microns.

11. The transdermal delivery device as claimed in claim 10, the cross-sectional area of the at least one channel proximate to the base of the microneedle being greater than or equal to about 0.5% of the total area of the base and being less than or equal to about 40% of the total area of the base.

12. The transdermal delivery device as claimed in claim 10, the total cross-sectional area of the channels on one microneedle, the cross-sectional areas taken proximate to the base of the microneedle, the total cross-sectional areas being less than or equal to about 40% of the total area of the base.

13. The transdermal delivery device as claimed in claim 10, the cross-sectional area of the at least one channel proximate to the base of the microneedle being greater than or equal to about 100 square microns.

14. The transdermal delivery device as claimed in claim 10 further comprising at least two microneedles, wherein the junctions on the at least two microneedles, when added together, have a total cross-sectional area that is greater than or equal to about 600 square microns.
